SEQUENCE LISTING (8 sequences)

SEQ ID NO: 1 - DNA, 29 bp, Homo sapiens
gccagctaggatccgtcgggatgtcacac

SEQ ID NO: 2 - DNA, 29 bp, Homo sapiens
tcgatcgcggccgctcagtacatgtccct

SEQ ID NO: 3 - DNA, 29 bp, Homo sapiens
gccagctaggatcccacaaactagtgcag

SEQ ID NO: 4 - DNA, 37 bp, Homo sapiens
tcgatcgaattcccatggctccagttgatcttaaatc

SEQ ID NO: 5 - DNA, 29 bp, Homo sapiens
gccagctaggatccctgggcatgtcacac

SEQ ID NO: 6 - DNA, 29 bp, Homo sapiens
tcgatcgcggccgcttagtacatgtcctt

SEQ ID NO: 7 - DNA, 399 bp, Homo sapiens
cacaaactagtgcagcttttgacaacaactgccgaacagcagttacggcatgctgatatagacacaagctgcaaagatgtcctgtcttgcacaggcacttccaactctgcctctgctaactcttcaggaggttcttgtccctcttctcatagctcattgacagaacggcataaaattctacaccggctcttacaggagggtagcccctcagatatcaccactttgtctgtcgagcctgataaaaaggacagtgcatctacttctgtgtcagtgactggacaggtacaaggaaactccagtataaaactagaactggatgcttcaaagaaaaaagaatcaaaagaccatcagctcctacgctatcttttagataaagatgagaaagatttaagatcaact

SEQ ID NO: 8 - DNA, 1814 bp, Photinus pyralis
ccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgtatacctctggatccactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgctcggaggagttgtgtttgtggacgaagtaccgaaaggtcttaccggaaaactcgacgcaagaaaaatcagagagatcctcataaaggccaagaagggcggaaagtccaaattgtaaaatgtaactagtattcagcgatgacgaaattcttagctattgtaatattatatgcaaattgatgaatggtaattttgtaattgtgggtcactgtactattttaacgaataataaaatcaggtataggtaactaaaaagaattcgagctcggtacccggg

(19) United States
(12) Patent Application Publication
(10) Pub. No.: US 2003/0039980 A1
(43) Pub. Date: Feb. 27, 2003

(54) Assays for determination of functional binding of compounds to receptors

(76) Inventor: John F. Thompson, Warwick, RI (US)

Correspondence Address: Gregg C. Benson, Pfizer Inc., Patent Department, MS 4159, Eastern Point Road, Groton, CT, 06340, US

(21) Appl. No.: 09/967,107

(22) Filed: Sep. 28, 2001

Related U.S. Application Data
(60) Provisional application No. 60/237,544, filed on Sep. 30, 2000.

(51) Int. Cl. C12Q 1/68; G01N 33/53

(52) U.S. Cl. 435/6; 435/7.1

(57) ABSTRACT

The present invention relates to novel processes for determination of functional binding of agents to receptors. The invention provides assays that measure the ligand-dependent interaction between nuclear receptors and nuclear receptor coregulators, including coactivators and corepressors. The invention further provides assays that can measure the ability of test agents to act as effectors of such interactions. The invention also provides pharmaceutical compositions comprising agents identified using the assays of the invention.

### CROSSREFERENCE TO RELATED APPLICATION

This application claims priority from U.S. Provisional Patent Application No. 60/237,544 filed Sep. 30, 2000, the benefit of which is hereby claimed under 37 C.F.R. §1.78(a)(3).

### FIELD OF THE INVENTION

The present invention relates to novel processes for determination of functional binding of agents to receptors. More specifically, the invention provides assays that measure the ligand-dependent interaction between nuclear receptors and nuclear receptor coregulators, including coactivators and corepressors. The invention further provides assays that can measure the ability of test agents to act as agonists or antagonists of nuclear receptors by affecting, for example, the ligand-dependent interaction between nuclear receptors and nuclear receptor coregulators, including coactivators and corepressors. The invention also provides pharmaceutical compositions comprising agents identified using the assays of the invention.

### BACKGROUND OF THE INVENTION

Nuclear receptors are ligand-inducible transcription factors that mediate numerous physiological roles. Together, they form a superfamily which comprises the largest known family of eukaryotic transcription factors.

A first class of nuclear receptors is type I, or steroid hormones, which comprises, inter alia, peroxisome proliferator-activated receptors (PPAR), estrogen receptors (ER), progestin receptors(PR), androgen receptors (AR), glucocorticoid receptors (GR), and mineralcorticoid receptors (MR). Type I nuclear receptors are associated with heat-shock proteins and are sequestered in the cytoplasm in the inactive state. Upon ligand binding, they dissociate from the heat shock proteins, homodimerize, translocate to the nucleus and bind to their specific DNA elements where they modulate transcription.

A second class of nuclear receptors is type II, or non-steroid hormones, which comprises, inter alia, all-trans retinoic acid receptors (RAR), thyroid hormone receptors (TR), and vitamin D receptors (VDR). Type II nuclear receptors remain strictly nuclear, heterodimerize with the receptor for 9-cis retinoic acid (RXR) and are constitutively bound to their target DNA elements.

A third class of nuclear receptors is the orphan receptors for which no endogenous ligands have yet been identified.

Nuclear receptors contain a number of conserved domains named A to F. The A/B region is weakly conserved and contains an autonomous activation function (AF-1). The C domain contains two Zinc-finger-like motifs which mediate DNA binding. The D domain is a variable hinge. The E Domain comprises the ligand-binding domain (LBD), a second activation function (AF-2), a dimerization domain, and a nuclear localization signal. Domain F has no known function.

Nuclear receptors in their active state function as either transcriptional activators or repressors. They associate in a ligand-dependent manner with coregulators, either coactivators or corepressors, and mediate transcription either by modulating the activities of the basal transcriptional apparatus or by chromatin remodeling.

Nuclear receptor coactivators include steroid receptor coactivator-1 (SRC-1), steroid receptor coactivator-2 (SRC-2), steroid receptor coactivator-3 (SRC-3), transcription intermediary factor 2 (TIF2), glucocorticoid receptor interacting protein 1 (GRIP1), retinoic acid receptor interacting protein 3 (RAC3), coactivator-associated arginine methyltransferase 1 (CARM1), peroxisome proliferator-activated receptor gamma coactivator-1 (PGC-1), peroxisome proliferator-activated receptor gamma coactivator-2 (PGC-2), p300, CREB binding protein (CBP), p300/CREB-binding protein-interacting protein (p/CIP), p300/CBP-associated factor (P/CAF), nuclear-receptor co-activator (NCoA) proteins, alteration/deficiency in activation (ADA) 3 protein, small nuclear RING finger protein (SNURF), the thyroid hormone receptor-associated proteins (TRAP), and NR-binding SET-domain-containing protein (NSD1).

Nuclear receptor corepressors include nuclear receptor corepressor (N-Cor), small ubiquitous nuclear corepressor (SUN-Cor), silencing mediator for retinoic acid and thyroid hormone receptors (SMRT), TIF2, thyroid hormone receptor uncoupling protein (TRUP), calreticulin, repressor of estrogen receptor activity (REA), and NR-binding SET-domain-containing protein (NSD1).

Nearly all factors that have been identified by their ability to interact with nuclear receptors in a ligand-dependent manner contain one or more copies of the LXXLL motif where L is leucine and X is any amino acid. The LXXLL motif has been shown to be the surface on the nuclear receptor coregulators that contacts the nuclear receptors. Upon binding of ligand to a nuclear receptor, the nuclear receptor's AF-2 helix undergoes conformational changes that result in a surface that interacts with the LXXLL motif of one or more coregulators. This conformational change and resulting interaction is an important target for pharmaceutical compositions comprising agonists or antagonists of nuclear receptor function.

The techniques of the prior art for measuring the ligand-dependent interaction of nuclear receptors with their coregulators has been laborious, inefficient and ill-suited for the assays required by many current drug discovery laboratories.

In one technique found in the prior art, a nuclear receptor LBD is expressed in bacteria as a fusion protein with glutathione-S-transferase (GST). A radioactively-labeled coactivator is then allowed to interact with the nuclear receptor LBD and the complex is then isolated by the interaction between the GST and glutathione affixed to a solid support. Then the complex is subjected to SDS-PAGE and autoradiography. This technique requires costly and hazardous radioisotopes as well as multiple labor-intensive steps which prevents its use as a basis for high-throughput assays.

In another technique found in the prior art, a nuclear receptor is labeled with a fluorescent reagent and a nuclear receptor coactivator is labeled with another, spectroscopically-complementary fluorescent reagent. To detect an interaction between the receptor and coactivator, the two fluorescent reagents must come in close enough proximity to allow a fluorescence resonance energy transfer from one to the other. Thus, the effectiveness of a putative agonist or antagonist can be measured on specialized equipment by any changes in the ratio of fluorescence of the two reagents resulting from the energy transfer between them.

The prior art has enabled this technique using essentially fluorescent reagents comprising europium cryptate or other lanthanide elements combined with the spectroscopically-complementary fluorescent reagent XL665. The advantage of fluorescent reagents comprising europium cryptate or other lanthanide elements is that their molecular structure limits background noise. This technique, however, is limited by (1) any difficulties in obtaining the correct juxtaposition of the fluorescent label on the nuclear receptors and their coactivators; (2) any difficulties in actually labeling the nuclear receptors and their coactivators; (3) expensive reagents; and (4) the necessity of purchasing specialized equipment. Moreover, if other, more conventional fluorescent reagents are used, unacceptable background noise might result.

Therefore, the art lacks a simple, inexpensive and reliable basis for a high-throughput assay for measuring the ligand-dependent interaction between a nuclear receptor and its coregulators. Such a high-throughput assay system would enable the art to identify ligands and functional agonists or antagonists of the nuclear receptor.

### SUMMARY OF THE INVENTION

The present invention relates to assays for the determination of functional binding of agents to receptors. More specifically, the invention provides assays that measure the ligand-dependent interaction between nuclear receptors and nuclear receptor coregulators, including coactivators and corepressors. The invention further provides assays that can measure the ability of a test agent(s) to act as an agonist(s) or an antagonist(s) of nuclear receptors by affecting the ligand-dependent interaction between nuclear receptors and nuclear receptor coregulators, including coactivators and corepressors. The invention also provides pharmaceutical compositions comprising an effector and a pharmaceutically acceptable carrier, vehicle, or diluent.

Accordingly, the present invention provides methods for the determination of the functional effects of test agents on nuclear receptor proteins or active fragments thereof, comprising the steps of:

A. combining in a first in vitro reaction cocktail a nuclear receptor protein or an active fragment thereof fused to a purification facilitating compound; a nuclear receptor coregulator protein or an active fragment thereof fused to an enzyme or a fragment thereof whose activity is simply quantified; a ligand for the nuclear receptor protein; a purification facilitating partner affixed to a solid support; and the test agent;

B. incubating the components of step (A) to allow said components to form a complex;

C. removing the solid support from the remainder of the first in vitro reaction cocktail;

D. determining an amount of the complex that was formed by assaying the removed solid support for a first activity of the enzyme or fragment thereof; and

E. assessing whether the test agent functioned as an effector of complex formation by comparing the first activity to a second activity from the enzyme or fragment thereof, recovered from a second in vitro reaction cocktail comprising all of the components of step A except the test agent, where the second in vitro reaction cocktail was subjected to steps (B) to (D).

The invention also provides high-throughput assays comprising methods described herein and utilizing multiple in vitro reaction cocktails for the determination of the functional effect of multiple test agents on a nuclear receptor protein or a fragment thereof.

The invention further provides methods for the identification of nuclear receptor ligands, comprising the steps of:

A. combining in a first in vitro reaction cocktail a nuclear receptor protein or an active fragment thereof fused to a purification facilitating compound; a nuclear receptor coregulator protein or an active fragment thereof fused to an enzyme or a fragment thereof whose activity is simply quantified; a purification facilitating partner affixed to a solid support; and a test agent;

B. incubating the components of step (A) to allow the components to form a complex;

C. removing said solid support from the remainder of the first in vitro reaction cocktail;

D. determining an amount of complex that was formed by assaying the removed solid support for a first activity of the enzyme or fragment thereof; and

E. assessing whether the test agent functioned as a ligand for the nuclear receptor or active fragment thereof by comparing the first activity to a second activity from the enzyme or fragment thereof recovered from a second in vitro reaction cocktail comprising all of the components of step (A) except the test agent, where the second in vitro reaction cocktail was subjected to steps (B) to (D).

The invention further yet provides high-throughput assays comprising the methods of the current invention and utilizing multiple in vitro reaction cocktails for the identification of ligands for nuclear receptor proteins or fragments thereof.

The invention also provides methods for the determination of functional effects of test agents on a peroxisome proliferator-activated receptor protein or a fragment thereof, comprising the steps of:

A. combining in a first in vitro reaction cocktail the peroxisome proliferator-activated receptor protein or a fragment thereof fused to the glutathione-S-transferase protein or a fragment thereof; a steroid receptor coactivator-1 protein or a fragment thereof fused to the luciferase protein or a fragment thereof; a GW2331 ligand; glutathione-sepharose beads; and the test agent;

B. incubating the components of step (A) to allow the components to form a complex;

C. removing the glutathione-sepharose beads from the remainder of the first in vitro reaction cocktail;

D. determining an amount of the complex that was formed by assaying the removed glutathione-sepharose beads for a first luciferase activity; and

E. assessing whether the test agent functioned as an effector of complex formation by comparing the first luciferase activity to a second luciferase activity recovered from a second in vitro reaction cocktail comprising all of the components of step A except the test agent, where the second in vitro reaction cocktail was subjected to steps (B) to (D).

The invention additionally provides methods for the identification of ligands for a peroxisome proliferator-activated receptor protein or a fragment thereof, comprising the steps of:

A. combining in a first in vitro reaction cocktail the peroxisome proliferator-activated receptor protein or a fragment thereof fused to the glutathione-S-transferase protein or a fragment thereof; a steroid receptor coactivator-1 protein or a fragment thereof fused to the luciferase protein or a fragment thereof; glutathione-sepharose beads; and a test agent;

B. incubating the components of step (A) to allow the components to form a complex;

C. removing the glutathione-sepharose beads from the remainder of the first in vitro reaction cocktail;

D. determining an amount of the complex that was formed by assaying the removed glutathione-sepharose beads for a first luciferase activity; and

E. assessing whether the test agent functioned as a ligand for peroxisome proliferator-activated receptor protein by comparing the first luciferase activity to a second luciferase activity recovered from a second in vitro reaction cocktail comprising all of the components of step A except the test agent, where the second in vitro cocktail was subjected to steps (B) to (D).

In a preferred embodiment, the test agents provided by the invention include, but are not limited to, proteins, peptides, nucleic acids, hormones, cytokines, lipids, carbohydrates, vitamins, minerals, large organic molecules, small organic molecules, non-organic agents or any combination thereof.

In another preferred embodiment, the nuclear receptors provided by the invention include steroid receptors and non-steroid receptors. In a more preferred embodiment, the nuclear receptors provided by the invention include, but are not limited to, the list comprising peroxisome proliferator-activated receptor, thyroid receptor, estrogen receptor, glucocorticoid receptor, progesterone receptor, androgen receptor, mineralcorticoid receptor, retinoic acid receptor, retinoid X receptor, vitamin D receptor, orphan receptors, any fragment thereof or any combination thereof.

In yet another preferred embodiment, active fragments of the nuclear receptor comprise the ligand binding domain. In yet a further preferred embodiment, such active fragments of the nuclear receptor coregulator comprise one or more LXXLL motifs.

In another preferred embodiment, the purification facilitating compounds provided by the invention include, but are not limited to, glutathione-S-transferase, maltose K, influenza hemaglutinin, avidin, biotin, FLAG, myc tag, histidine multimers, or any combination thereof.

In another preferred embodiment, the purification facilitating partners provided by the invention include, but are not limited to, glutathione, maltose, anti-influenza hemaglutinin antibodies, avidin, biotin, anti-FLAG antibodies, anti-myc antibodies, ionic nickel, or any combination thereof.

In yet another preferred embodiment, the nuclear receptor coregulators provided by the invention include nuclear receptor coactivators. In a more preferred embodiment, the nuclear receptor coactivators provided by the invention include, but are not limited to, steroid receptor coactivator-1, steroid receptor coactivator-2, steroid receptor coactivator-3, transcription intermediary factor 2, glucocorticoid receptor interacting protein 1, retinoic acid receptor interacting protein 3, coactivator-associated arginine methyltransferase 1, peroxisome proliferator-activated receptor gamma coactivator-1, peroxisome proliferator-activated receptor gamma coactivator-2, p300/CREB binding protein, p300, CREB-binding protein-interacting protein, nuclear-receptor co-activator protein, p300/CBP-associated factor, alteration/deficiency in activation 3 protein, small nuclear RING finger protein, thyroid hormone receptor-associated protein 220, NR-binding SET-domain-containing protein, any fragment thereof, or any combination thereof.

In another preferred embodiment, the nuclear receptor coregulators provided by the invention include nuclear receptor corepressors. In a more preferred embodiment, the nuclear receptor corepressors provided by the invention include, but are not limited to, nuclear receptor corepressor, small ubiquitous nuclear corepressor, silencing mediator for retinoic acid and thyroid hormone receptors, transcription intermediary factor 2, thyroid hormone receptor uncoupling protein, calreticulin, repressor of estrogen receptor activity, NR-binding SET-domain-containing protein, any fragment thereof, or any combination thereof.

In a preferred embodiment, the solid support is a glass bead, cellulose bead, polystyrene bead, sephadex bead, sepharose bead, polyacrylamide bead, agarose bead, magnetic bead, multi-well plate, glass reaction vessel, or plastic reaction vessel.

In a preferred embodiment, the enzyme is luciferase, β-galactosidase, alkaline phosphatase, peroxidase, chloramphenicol acetyl transferase or green fluorescent protein. In another preferred embodiment, a detectable agent may be used to detect complex formation. Such agents include, but are not limited to, light emitting agents, fluorescent agents, radiolabels, affinity labels, and known antigens.

In a preferred embodiment, the second in vitro reaction cocktail comprises a control agent known to have an effect on the nuclear receptor.

In a preferred embodiment, the first in vitro reaction cocktail comprises multiple nuclear receptors or active fragments thereof essentially simultaneously and the method comprises an additional step (F) comprising deconvoluting the active nuclear receptor after assessing whether the test agent functioned as an effector of the nuclear receptor protein.

In a preferred embodiment, the test agent functioned as an agonist of complex formation, an antagonist of complex formation, or a ligand for the nuclear receptor protein.

In a preferred embodiment, the second in vitro reaction cocktail comprises a control agent known to be a ligand for the nuclear receptor or active fragment thereof.

Unless otherwise noted, the terms used throughout this specification and the appendant claims generally have their usual meaning as understood by those of ordinary skill in the art. The following terms are intended to have the following general meanings as they are used herein:

“active fragment” refers to a portion of a protein which retains an activity of interest. An active fragment of a nuclear receptor refers to any fragment of the nuclear receptor that is capable of binding to a ligand of the nuclear receptor and a coregulator wherein the binding to the coregulator occurs in a ligand-dependent fashion. Thus, in a preferred embodiment, an active fragment comprises the nuclear receptor ligand binding domain. An active fragment of a nuclear receptor coregulator refers to any fragment of the coregulator that is capable of binding to the nuclear receptor in a ligand-dependent fashion. In a preferred embodiment, an active fragment of a nuclear receptor coregulator comprises one or more LXXLL motifs. For the purposes of the assay as provided by the invention, an active fragment of a nuclear receptor coregulator may, but is not required to, retain a transcriptional modulatory function;

“affinity label” refers to a first agent that, when attached to a protein of interest, e.g. a nuclear receptor coregulator, results in that protein of interest having an affinity for a second, detectable agent via the attached first agent. These first and second agents operate much like the purification facilitating proteins and partners described below, but are not affixed to solid supports and function in the detection, rather than purification, of nuclear receptor complexes. Affinity labels may be chosen, e.g., from the list described in the purification facilitating protein and partner definitions described below;

“agent” refers to any molecule, element or compound that has a functional effect on a nuclear receptor. For example, agents can be either organic or inorganic and may function as an agonist, antagonist or ligand to a nuclear receptor, a fragment thereof, or a complex thereof. A “test agent” refers to any agent assessed by the assays provided by the current invention for a functional effect on said nuclear receptor, fragment or complex. A “control agent” refers to any agent having a known effect on the nuclear receptor used in the assays provided by the current invention for the purpose of comparison with the effect of a test agent;

“agonist” refers to any agent identified in the assay provided by the invention that facilitates or promotes the activity or function of a nuclear receptor. The agonist may function by promoting the ligand-dependent interaction of a nuclear receptor with a nuclear receptor coregulator;

“antagonist” refers to any agent identified in the assay provided by the invention that interferes with the activity or function of a nuclear receptor. The antagonist may function by interfering with the ligand-dependent interaction of a nuclear receptor with a nuclear receptor coregulator;

“coactivators” refer to proteins which bind to nuclear receptors in a ligand-dependent fashion and facilitate the activation of target genes by the nuclear receptors to which they are bound;

“coregulators” refer to proteins which bind to nuclear receptors in a ligand-dependent dependent fashion and facilitate the activation or repression of target genes by the nuclear receptor to which they are bound. Coregulators comprise both coactivators, corepressors, and molecules that function as both a coactivator and a corepressor;

“corepressors” refer to proteins which bind to nuclear receptors in a ligand-dependent fashion and facilitate the repression of target genes by the nuclear receptors to which they are bound;

“deconvoluting the active receptor(s)” refers to the process of determining which of several receptors tested simultaneously in the same solution is affected by a test agent. After a test agent has been identified to be active against one or more nuclear receptors in a mixture of nuclear receptors, that agent can be examined in isolation against each of the individual nuclear receptors until it is determined against which of the nuclear receptors the test agent is active;

“effector” refers to any agonist, antagonist, ligand or other agent that affects the activity of the nuclear receptor used in the assays of the current invention. Effectors can be, but are not limited to, peptides, carbohydrates, nucleic acids, lipids, fatty acids, hormones, organic compounds, and inorganic compounds;

“enzyme whose activity is simply quantified” refers to any enzyme for which a quick, reliable, standardized assay exists which allows for accurate quantification of its activity. The art recognizes many such enzymes, including, but not limited to, firefly, bacterial and other luciferases, β-galactosidase, alkaline phosphatase, peroxidase, CAT, and green fluorescent protein;

“high-throughput assay” refers to an assay that can be partially or fully automated, allowing for multiple in vitro reaction cocktails to be assayed essentially simultaneously for a functional effect of multiple test agents on a nuclear receptor molecule or an active fragment thereof. A high-throughput assay may also comprise multiple in vitro reaction cocktails to be assayed essentially simultaneously for a functional effect of a single test agent on multiple nuclear receptor molecules, active fragments thereof, or any suitable combination thereof;

“ligand” refers to any molecule that binds to a nuclear receptor. Typically, nuclear receptor ligands are hormones, vitamins, fatty acids, proteins or steroids. However, the term “ligand” as used herein may also refer to other organic or non-organic molecules that bind with specificity to a nuclear receptor. Ligands may be naturally occurring or synthetic and upon binding often affect nuclear receptor function;

“ligand binding domain (LBD)” refers to a domain of nuclear receptors that bind nuclear receptor ligands and mediate the effects of ligand binding on nuclear receptor function;

“LXXLL motif” refers to an evolutionarily-conserved amino acid motif that occurs one or more times on nuclear coregulator proteins. The LXXLL motif mediates the protein-protein interactions between a nuclear receptor and a nuclear receptor coregulator, where “L” refers to the amino acid leucine and “X” refers to any amino acid;

“nuclear receptors” refers to a family of eukaryotic transcription factors that localize in the cytoplasm or the nucleus of a cell and are activated by binding to a ligand. Once activated by ligand binding, nuclear receptors specifically bind to cis DNA elements of target genes and either activate or repress transcription therefrom;

“pharmaceutical composition” refers to any composition comprising an agent(s) provided by the present invention formulated in any suitable fashion such as any suitable formulation well known in the art and a pharmaceutically acceptable carrier, vehicle, or diluent. Any suitable route of administration of the pharmaceutical composition may be used including, for example, injection, transmucosal, oral, inhalation, ocular, rectal, long acting implantation, liposomes, emulsion, and sustained release means;

“purification facilitating partner” refers to any agent which can be affixed to a solid support for the purposes of facilitating the purification of a nuclear receptor via its binding to a purification facilitating compound fused to the nuclear receptor. The purification facilitating partner may be, but is not limited to, a compound or protein chosen from the following list: glutathione (GSH), Maltose, anti-influenza hemaglutinin (HA) antibodies, avidin, biotin, anti-FLAG antibodies (Sigma-Aldrich), anti-myc antibodies, and ionic nickel;

“purification facilitating compound” refers to any compound which, when fused to the nuclear receptor by any suitable means, e.g., recombinant DNA techniques, chemical fusion, biochemical fusion or immunohistochemical fusion, facilitates the nuclear receptor's purification from any medium or solution, including an in vitro reaction cocktail, via its binding to a purification facilitating partner affixed to a solid support. The purification facilitating compound may be, but is not limited to, a compound chosen from the following list: glutathione-S-transferase (GST), Maltose K (MalK), influenza hemaglutinin (HA), avidin, biotin, FLAG (Sigma-Aldrich), myc tag and histidine multimers;

“solid support” refers to any surface or non-soluble substance which can form the basis for simple physical separation of an in vitro reaction component from the remainder of an in vitro reaction cocktail. The solid support may either be added to the reaction cocktail or may comprise the walls of the reaction vessel. Many such solid supports are known in the art, and include, but are not limited to, glass beads, cellulose beads, polystyrene beads, sephadex beads, sepharose beads, polyacrylamide beads, agarose beads, magnetic beads, multi-well plates, glass reaction vessels and plastic reaction vessels;

“test agent” refers to any substance added to the assay provided by the invention for the purpose of determining whether it has agonistic or antagonistic properties on the nuclear receptor being assayed. Test agents can be, but are not limited to, the group comprising proteins, peptides, nucleic acids, hormones, cytokines, lipids, carbohydrates, vitamins, minerals, large organic molecules, small organic molecules, non-organic agents and any combination thereof;

“transcriptional activators” refer to any of a class of proteins, e.g., those well known in the art, that increase the transcription of target genes by many different known mechanisms. Typically, transcriptional activators function by increasing transcriptional initiation, by increasing transcript elongation, or by affecting chromosomal remodeling. Transcriptional activators either bind to DNA or are drawn into multi-protein complexes through protein-protein interactions;

“transcriptional repressors” refer to any of a class of proteins, e.g., those well known in the art , that decrease the transcription of target genes by many different known mechanisms. Typically, transcriptional repressors function by competing with transcriptional activators for critical DNA or protein contacts, by directly decreasing transcriptional initiation, by directly decreasing transcript elongation, or by affecting chromosomal remodeling. Transcriptional repressors either bind with specificity to DNA or are drawn into multi-protein complexes through protein-protein interactions; and

° C. is degrees Centigrade; % is percent; DTT is dithiothreitol; EDTA is ethylenediaminetetraacetic acid; h is hour(s); KCI is potassium chloride; min is minute(s); mL is milliliter(s); mM is millimolar (concentration); NaCl is sodium chloride; nM is nanomolar (concentration); PBS is phosphate-buffered saline; rpm is revolutions per minute; SDS-PAGE is sodium dodecenyl sulfate-polyacrylamide gel electrophoresis; sec is second(s); μg is microgram(s); μL is microliter(s); μM is micromolar (concentration); and w/v is weight per volume.

Amersham Pharmacia Biotech is located in Uppsala, Sweden); DYNEX Technologies, Inc. is located at 14340 Sullyfield Circle, Chantilly, Va. 20151, U.S.A.); Novagen, Inc. is located at 601 Science Drive, Madison, Wis. 53711, U.S.A.); Promega Corp., is located in at 2800 Woods Hollow Road, Madison, Wis. 53711, U.S.A.); and Sigma-Aldrich is located at 1 Strathmore Road, Natick, Mass. 01760).

All of the documents cited herein are incorporated by reference herein in their entireties.

### BRIEF DESCRIPTION OF THE DRAWINGS

For a better understanding of the invention as well as other objects and further features thereof, reference is made to the following detailed description of various preferred embodiments thereof taken in conjunction with the accompanying drawings wherein:

FIG. 1 is a schematic representation of a preferred embodiment of the present invention. It depicts a GST-nuclear receptor-LBD fusion protein which interacts with a luciferase-coregulator fusion protein in a ligand-dependent manner. The complex is captured on a solid substrate, namely, glutathione beads.

FIG. 2 is a representation of the results of a preferred embodiment of the present invention. Specifically, this figure shows luciferase activities, measured in light units, from complexes isolated from in vitro reaction cocktails comprising GST-PPARα-LBD, SRC-Luc and spec/2-(4-[2-(3[2,4-Difluorophenyl]-1-heptylureido)ethyl]phenoxy)-2-methylbutyric acid (GW2331) ligand (see Kliewer S. A. et al., Proc. Natl. Acad. Sci. 94:4318-4323 (1997)). As those skilled in the art will understand from FIG. 2, as the concentration of GW2331 ligand was increased in each reaction cocktail, the amount of GST-PPARα-LBD/GW2331 ligand/SRC-Luc complex also increased.

FIG. 3 is a representation of the results of a preferred embodiment of the present invention. Specifically, this figure shows luciferase activity, measured in light units, from complexes isolated from in vitro reaction cocktails comprising GST-PPARα-LBD or GST-PPARβ-LBD, SRC-Luc and GW2331 or “Compound A” from International Application Publication No. WO 97/28149 (see also, U.S. Pat. Nos. 5,847,008; 5,859,051; 6,020,382; 6,090,836; 6,090,839; and 6,160,000; and International Application Publication Nos. WO97/27847, WO97/27857; WO97/28115; WO97/28137; WO98/27974), where each ligand was added to a final 5 μM oncentration.

### DETAILED DESCRIPTION OF THE INVENTION

Because nuclear receptors play an important role in many physiological processes and diseases, including cancer, diabetes, obesity, osteoporosis, frailty, cardiovascular disease, inflammation, cognitive disorders, and drug-drug interactions, there is a need in the art for a simple, reliable and inexpensive method for identifying agents that modulate nuclear receptor activity. Such methods would form the basis, for instance, of high-throughput assays to identify, e.g., ligands, agonists or antagonists, of nuclear receptors. Agents that have been found to function as, e.g., ligands, agonists or antagonists, of nuclear receptors might then be developed into drugs for the treatment of human or other animal diseases or conditions associated with the nuclear receptors.

Therefore, the invention described herein provide methods for the determination of functional effects of test agents on nuclear receptor proteins or fragments thereof, comprising the steps of:

A. combining in a first in vitro reaction cocktail the nuclear receptor protein or an active fragment thereof fused to a purification facilitating compound; a nuclear receptor coregulator protein or an active fragment thereof fused to an enzyme or a fragment thereof whose activity is simply quantified; a ligand for the nuclear receptor protein; a purification facilitating partner affixed to a solid support; and the test agent;

B. incubating the components of step (A) to allow the components to form a complex;

C. removing the solid support from the remainder of the first in vitro reaction cocktail;

D. determining an amount of complex that was formed by assaying the removed solid support for a first activity of the enzyme or fragment thereof; and

E. assessing whether the test agent functioned as an effector of complex formation by comparing the first activity to a second activity from the enzyme or fragment thereof recovered from a second in vitro reaction cocktail comprising all of the components of step (A) except the test agent, where said second in vitro reaction cocktail was subjected to steps (B) to (D).

In a preferred embodiment, the second in vitro reaction cocktail comprises a control agent known to have agonistic or antagonistic activity for the nuclear receptor or active fragment thereof.

In another preferred embodiment, the amount of the complex that is determined is a detectable amount of the complex. In another preferred embodiment, the amount of the complex that is determined is essentially all of the complex.

Moreover, the invention described herein provides methods for the identification of a nuclear receptor ligand, comprising the steps of:

A. combining in a first in vitro reaction cocktail the nuclear receptor protein or an active fragment thereof fused to a purification facilitating compound; a nuclear receptor coregulator protein or an active fragment thereof fused to an enzyme or a fragment thereof whose activity is simply quantified; a purification facilitating partner affixed to a solid support; and a test agent;

B. incubating the components of step (A) to allow the components to form a complex;

C. removing the solid support from the remainder of the first in vitro reaction cocktail;

D. determining an amount of complex that was formed by assaying the removed solid support for a first activity of the enzyme; and

E. assessing whether the test agent functioned as a ligand for the nuclear receptor or active fragment thereof by comparing the first activity to a second activity from the enzyme or a fragment thereof recovered from a second in vitro reaction cocktail comprising all of the components of step (A) except the test agent, where the second in vitro reaction cocktail was subjected to steps (B) to (D).

In a preferred embodiment, the second in vitro reaction cocktail comprises a control agent known to be a ligand for the nuclear receptor or active fragment thereof.

In another preferred embodiment, the amount of the complex that is determined is a detectable amount of the complex. In another preferred embodiment, the amount of the complex that is determined is essentially all of the complex.

In another preferred embodiment, the nuclear receptor and the nuclear receptor coregulator, or their respective active fragments, were at no time fused to the same purification facilitating compound prior to the assay provided by the invention.

In each of the methods of this invention, the step of removing the solid support from the remainder of the reaction cocktail refers to either removal of the solid support from the reaction vessel or removal of the remaining reaction cocktail ingredients from the reaction vessel. Preferably, the solid support is washed to remove any non-specific binding of the coregulator fusion product before determining the amount of complex formed on the solid support.

The invention, where used as a high-throughput assay, may be used to screen a large number of substances to identify nuclear receptor ligands, agonists or antagonists. Advances in organic chemistry, combinatorial chemistry, biochemistry and molecular biology have provided the art with libraries of agents and substances, either natural, recombinant or synthetic, that can be used as test agents in the assays provided by the invention. In a preferred embodiment, the test agent of the invention is chosen from the list comprising proteins, peptides, nucleic acids, hormones, cytokines, lipids, carbohydrates, vitamins, minerals, large organic molecules, small organic molecules, non-organic agents and any combination thereof.

In a preferred embodiment, the nuclear receptor is either a steroid receptor or a non-steroid receptor.

In another preferred embodiment, the nuclear receptor is chosen from the list comprising peroxisome proliferator-activated receptors (PPAR), estrogen receptors (ER), progestin receptors(PR), androgen receptors (AR), glucocorticoid receptors (GR), mineralcorticoid receptors (MR), all-trans retinoic acid receptors (RAR), 9-cis retinoic acid receptor (RXR), thyroid hormone receptors (TR), vitamin D receptors (VDR), orphan receptors, any fragment thereof and any combination thereof.

Because the nuclear receptor ligand binding domain mediates ligand binding, coregulator binding, and nuclear receptor activation, the assays provided by the invention may be carried out using a truncated nuclear receptor comprising the ligand binding domain. Therefore, in a preferred embodiment, the fragment of the nuclear receptors provided by the invention comprises the ligand binding domain.

In another preferred embodiment, the invention provides high-throughput assays comprising multiple in vitro reaction cocktails for the determination of the functional effect of multiple test agents on a nuclear receptor protein or a fragment thereof.

In another preferred embodiment, the invention provides high-throughput assays comprising multiple in vitro reaction cocktails for the identification of a ligand for a nuclear receptor protein or a fragment thereof.

In another preferred embodiment, the invention provides high-throughput assays comprising multiple in vitro reaction cocktails for the determination of a functional effect of a single test agent or ligand on multiple nuclear receptors or fragments thereof.

In the course of running multiple assays or high-throughput assays, many or most test agents may be inactive on the nuclear receptor or receptors being tested. To minimize costs and to maximize testing efficiency, multiple receptors can be tested against each test agent simultaneously in the same in vitro reaction cocktail. When an agent is found to be active against the mixture of nuclear receptors, that agent can be examined in isolation against each of the individual nuclear receptors until it is determined which is the active nuclear receptor. Therefore, in another preferred embodiment, the invention provides the first in vitro reaction cocktail to comprise multiple nuclear receptors or active fragments thereof simultaneously and the methods provided by the invention to further comprise deconvoluting the active receptor after assessing whether the test agent functioned as an agonist, antagonist or ligand of one or more of the nuclear receptor proteins in the in vitro reaction cocktail.

In another preferred embodiment, the nuclear receptor coregulator provided by the invention is a nuclear receptor coactivator. In a more preferred embodiment, the nuclear receptor coactivator is chosen from the list comprising a steroid receptor coactivator-1 (SRC-1), steroid receptor coactivator-2 (SRC-2), steroid receptor coactivator-3 (SRC-3), transcription intermediary factor 2 (TIF2), glucocorticoid receptor interacting protein 1 (GRIP1), retinoic acid receptor interacting protein 3 (RAC3), coactivator-associated arginine methyltransferase 1 (CARM1), peroxisome proliferator-activated receptor gamma coactivator-1 (PGC-1), peroxisome proliferator-activated receptor gamma coactivator-2 (PGC-2), p300, CREB binding protein (CBP), p300/CREB-binding protein-interacting protein (p/CIP), nuclear-receptor co-activator (NCoA) proteins, p300/CBP-associated factor (P/CAF), alteration/deficiency in activation (ADA) 3 protein, small nuclear RING finger protein (SNURF), the thyroid hormone receptor-associated proteins (TRAP), NR-binding SET-domain-containing protein (NSD1), any fragment thereof and any combination thereof.

In another preferred embodiment, the nuclear receptor coregulator is a nuclear receptor corepressor. In a more preferred embodiment, the nuclear receptor corepressor is chosen from the list comprising nuclear receptor corepressor (N-Cor), small ubiquitous nuclear corepressor (SUN-Cor), silencing mediator for retinoic acid and thyroid hormone receptors (SMRT), TIF2, thyroid hormone receptor uncoupling protein (TRUP), calreticulin, repressor of estrogen receptor activity (REA), NR-binding SET-domain-containing protein (NSD1), any fragment thereof and any combination thereof.

Solid supports for in vitro assays, including the solid supports useful in the present invention, are well known in the art. In a preferred embodiment, the solid support provided by the invention is chosen from the list comprising glass beads, cellulose beads, polystyrene beads, sephadex beads, sepharose beads, polyacrylamide beads, agarose beads, magnetic beads, multi-well plates, glass reaction vessels and plastic reaction vessels. In a more preferred embodiment, the invention provides that a purification facilitating partner be affixed to the solid support so that any complex that has formed and includes a purification facilitating compound can be separated from the remainder of the in vitro reaction cocktail by mechanical, magnetic, or other physical means.

A critical component of the assay provided by the invention is a detectable agent or an enzyme whose activity is simply and accurately quantified. Such suitable agents and enzymes are well known in the art. In a preferred embodiment, the enzyme provided by the invention is chosen from the list comprising firefly, bacterial or other luciferases, β-galactosidase, alkaline phosphatase, peroxidase, CAT and green fluorescent protein. In another preferred embodiment, the detectable agent is chosen from the list comprising light emitting agents, fluorescent agents, radiolabels, affinity labels and known antigens. The enzymatic activity may be quantified by a luminometer, spectrophotometer, or any other means for measuring light or fluorescense absorption, scattering or emission. Preferably, the measuring means is an instrument that is commonly found in drug-discovery laboratories or may be purchased inexpensively.

In a more preferred embodiment, the invention provides methods for the identification of a ligand for a peroxisome proliferator-activated receptor protein or a fragment thereof, comprising the steps of:

A. combining in a first in vitro reaction cocktail the peroxisome proliferator-activated receptor protein or a fragment thereof fused to glutathione-S-transferase protein or a fragment thereof; a steroid receptor coactivator-1 protein or a fragment thereof fused to luciferase protein or a fragment thereof; glutathione-sepharose beads; and a test agent;

B. incubating the components of step (A) to allow the components to form a complex;

C. removing the glutathione-sepharose beads from the remainder of the first in vitro reaction cocktail;

D. determining an amount of said complex that was formed by assaying the removed glutathione-sepharose beads for a first luciferase activity; and

E. assessing whether the test agent functioned as a ligand for peroxisome proliferator-activated receptor protein by comparing the first luciferase activity to a second luciferase activity recovered from a second in vitro reaction cocktail comprising all of the components of step (A) except the test agent, where the second in vitro reaction cocktail was subjected to steps (B) to (D).

In a preferred embodiment, the second in vitro reaction cocktail comprises the peroxisome proliferator-activated receptor protein ligand GW2331 as a control agent.

In another preferred embodiment, the amount of the complex that is determined is a detectable amount of the complex. In another preferred embodiment, the amount of the complex that is determined is essentially all of the complex.

In another more preferred embodiment, the invention provides methods for the determination of the functional effect of a test agent on a peroxisome proliferator-activated receptor protein or a fragment thereof, comprising the steps of:

A. combining in a first in vitro reaction cocktail the peroxisome proliferator-activated receptor protein or a fragment thereof fused to glutathione-S-transferase protein or a fragment thereof; a steroid receptor coactivator-1 protein or a fragment thereof fused to the luciferase protein or a fragment thereof; GW2331 ligand; glutathione-sepharose beads; and the test agent;

B. incubating the components of step (A) to allow the components to form a complex;

C. removing the glutathione-sepharose beads from the remainder of the first in vitro reaction cocktail;

D. determining an amount of said complex that was formed by assaying the removed glutathione-sepharose beads for a first luciferase activity; and

E. assessing whether the test agent functioned as an effector of complex formation by comparing the first luciferase activity to a second luciferase activity recovered from a second in vitro reaction cocktail comprising all of the components of step (A) except the test agent, where the second in vitro reaction cocktail was subjected to steps (B) to (D).

In a preferred embodiment, the amount of the complex that is determined is a detectable amount of the complex. In another preferred embodiment, the amount of the complex that is determined is essentially all of the complex.

Ultimately, any agents, e.g. agonists or antagonists, that are identified by the assay provided by the invention might be a candidate drug for the treatment of a disease or condition in humans or animals. Therefore, in a preferred embodiment, the invention provides a pharmaceutical composition comprising an agonist of nuclear receptor function identified by the assay provided by the invention. In a more preferred embodiment, the invention provides a pharmaceutical composition wherein said agonist promotes the ligand-dependant interaction of a nuclear receptor with a nuclear receptor coregulator.

In another preferred embodiment, the invention provides a pharmaceutical composition comprising an antagonist of nuclear receptor function identified by the assay provided by the invention. In a more preferred embodiment, the invention provides a pharmaceutical composition wherein said antagonist interferes with the ligand-dependant interaction of a nuclear receptor with a nuclear receptor coregulator.

In another preferred embodiment, the invention provides a ligand for a nuclear receptor identified by the assay provided by the invention.

Because the art recognizes many methods of administering pharmaceutical compositions to patients, a more preferred embodiment of the invention provides a pharmaceutical composition whose route of administration is injection, transmucosal, oral, inhalation, ocular, rectal, long acting implantation, liposomes, emulsion, or by sustained release means.

The pharmaceutical compositions may be manufactured using any suitable means such as, for example, by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically or pharmaceutically acceptable carriers (vehicles, or diluents) comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks'solution, Ringer's solution, or physiological saline buffer. For transmucosal administration, penetrants appropriate to the barrier to be permeated are used in the formulation. Such penetrants are generally known in the art. For ocular administration, suspensions in an appropriate saline solution are used as is well known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetra-fluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, such as sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

One type of pharmaceutical carrier for hydrophobic compounds of the invention is a cosolvent system comprising benzyl alcohol, a nonpolar surfactant, a water-miscible organic polymer, and an aqueous phase.

The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may be substituted for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a prolonged period of time. In one embodiment, the sustained-release capsules may release compounds for a period of time as long as 120 days or more. In a more preferred embodiment, the sustained-release capsules may release compounds for a period of time as long as 90 days. In another preferred embodiment, the sustained-release capsules may release compounds for a period of time as long as 60 days. In another preferred embodiment, the sustained-release capsules may release compounds for a period of time as long as 30 days. In another preferred embodiment, the sustained-release capsules may release compounds for a period of time as long as seven days. In another more preferred embodiment, the sustained-release capsules may release compounds for a period of time as long as 1 day. In another preferred embodiment, the sustained-release capsules may release compounds for a period of time less than one day.

Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the agents of the invention may be provided as salts with pharmaceutically acceptable counterions. Pharmaceutically acceptable salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

The present invention is illustrated by the following examples. The foregoing and following description of the present invention and the various embodiments are not intended to be limiting of the invention but rather are illustrative thereof. Hence, it will be understood by those skilled in the art that the invention is not limited to the specific details of these examples.

### EXAMPLE 1

### An in vitro PPARα/SRC-1 Binding Assay

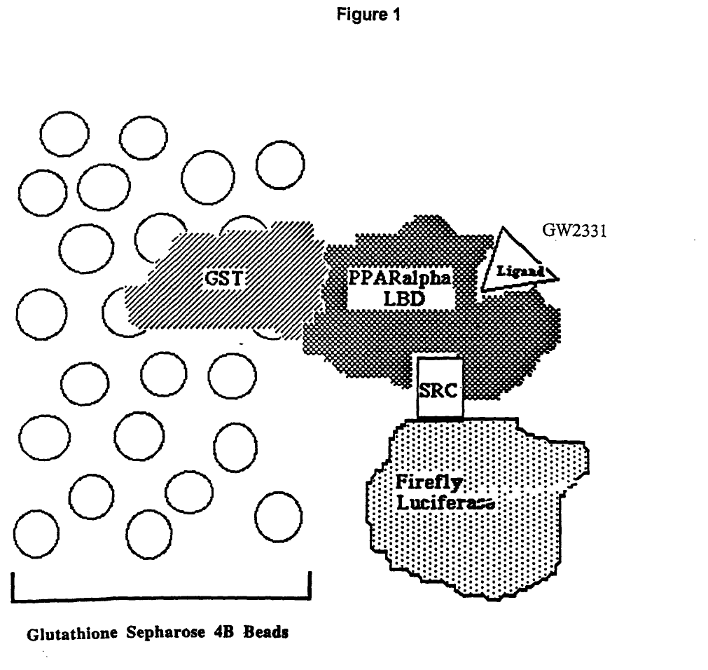

For this example, the peroxisome proliferator-activated receptor-α (PPARα) nuclear receptor was chosen. The PPAR LBD was expressed in bacteria as a GST fusion protein (GST-PPARα-LBD) for use in the assay (FIG. 1). Also chosen was a known PPARα coactivator, SRC-1 (Takeshita Endocrinology 137:3594-3597 (1996)). SRC-1 was expressed as a GST fusion protein also containing a firefly luciferase moiety (GST-SRC-Luc). The GST moiety was subsequently cleaved away from purified GST-SRC-1-Luc protein to yield a purified SRC-Luc protein (FIG. 1). A known PPARα ligand, GW2331, was also chosen for establishing the assay.

The GST-PPARα-LBD expression plasmid was prepared as follows: the human PPARα ligand binding domain including the hinge region (amino acids 164-468, accession #S74349) was PCR amplified from HepG2 first-strand cDNA using synthetic primers (SEQ ID:1 and SEQ ID:2) and native Taq polymerase. The primers were tagged with BamHI (5′) and NotI (3′) restriction sites to facilitate cloning. PCR fragments were cloned directly into pGEM®-T (Promega Corp), screened by diagnostic restriction digests, and confirmed by DNA sequence analysis. A combination of clones was used to generate a complete, error-free clone. The PPARα hinge/ligand binding domain clone was then transferred into pGEX-4T-3 (Amersham Pharmacia Biotech) as a BamH I/Not I fragment to create an in-frame fusion with GST.

The GST-SRC-Luc expression plasmid was prepared as follows: DNA encoding amino acids 631-763 from SRC-1 was PCR amplified using synthetic primers (SEQ ID:3 and SEQ ID:4) and native Taq polymerase. The primers were tagged with BamH I (5′) and Nco I/EcoR I (3′) sites to facilitate cloning and subsequent fusion with firefly luciferase. The SRC-1 PCR fragment was purified and digested with BamH I/EcoR I and cloned into BamH I/EcoR I-digested pGEX-6P-1 (Amersham Pharmacia Biotech) to create an in-frame fusion with GST. A positive, error-free clone (G6SRCC1) was identified by DNA sequence analysis. An Nco I/Sma I fragment containing the Photinus pyralis firefly cDNA (L194F/N197Y/S198T mutant, Thompson et al. J. Biol. Chem. 272:18766-18771 (1997), was transferred into Nco I/Sma I-digested G6SRCC1 to create a triple in-frame fusion of GST-SRC-Luc. Positive clones (G6SRCLuc-5, -8, and -17) were identified by screening colonies for firefly luciferase expression.

GST-PPARα-LBD and GST-SRC-Luc were separately expressed in E. coli as follows: Strain BL21(DE3)pLysS (Novagen, Inc.) was transformed with appropriate DNA and plated on LB plates with 100 μg/mL ampicillin. A single colony was used to inoculate 200 mL of L broth supplemented with 100 μg/mL ampicillin and grown to an optical density of 0.6 (600 nM). Cells were quickly cooled to room temperature followed by addition of IPTG to 50 μM and incubation overnight at room temperature. Cells were pelleted and resuspended in 5 ml of PBS (120 mM NaCl, 2.7 mM KCl, 10 mM phosphate pH 7.4) and frozen at −80° C. for 2 h. Cells were lysed by thawing at 37° C. for 15 min. 400 units/of DNase I and 250 μg of RNase were added and incubated until the viscosity decreased (about 10 min). Insoluble material was pelleted twice by spinning at 10,000 rpm for 10 min. 200 μg of glutathione sepharose 4B was added to the soluble fraction and incubated at room temperature with agitation for 30 min. After binding of the GST-SRC-Luc or the GST-PPARα-LBD to the GSH-sepharose beads, the complex was then transferred to a column, drained, and washed three times with 1 mL PBS at 0° C. After washing, the GSH-sepharose-GST-SRC-Luc or GSH-sepharose-GST-PPARα-LBD complex was resuspended in 400 μL PBS with 10 mM DTT and stored cold.

The GSH-sepharose-GST-SRC-Luc was cleaved to release the SRC-Luc protein from the GSH-sepharose-GST complex. SRC-Luc was cleaved and purified as follows: After the final PBS wash of GSH-sepharose-GST-SRC-Luc complex, 100 μL of beads were suspended in 100 μL of cleavage buffer (8 units precision protease, 10 μM DTT, 50 mM Tris-HCl (pH 7.0), 150 mM NaCl, 1 mM EDTA) and incubated overnight at 4° C. The beads were then placed in a column, drained, and washed with 200 μL of PBS. The collected eluate and wash were combined and stored cold.

GST-PPARα-LBD was not purified from the GSH-sepharose beads but was instead stored as a bound complex. The assay, as provided by the invention, normally would involve attaching the GST-PPARα-LBD to GSH-sepharose beads. Protocols for attaching GST fusion proteins to GSH-sepharose are generally known in the art, as are those conditions for adapting the assays provided by the invention to 96 well plates or other solid supports for use in high throughput screening.

The first step of the assay was to bind SRC-Luc to GST-PPARα-LBD in the presence of increasing concentrations of PPARα ligand (FIG. 1). One mL reactions were set up in 1.5 mL microcentrifuge tubes. This includes NETN (20 mM Tris-HCl (pH 8.0), 100 mM NaCl, 1 mM EDTA, 0.5% NP40), 1 μL SRC-Luc, and 5 μL GSH-sepharose beads with GST-PPARα-LBD attached. The PPARα ligand (GW2331) was added to separate in vitro binding cocktails to final concentrations of 0.001 μM, 0.01 μM, 0.1 μM, 1.0 μM, 10.0 μM and 100.0 μM. These mixtures were gently rocked for 2 h at 4° C. Following the incubation, beads were spun in a microfuge for 5-10 sec and the supernatant aspirated. The beads were washed four times by resuspending them in 400 μL cold NETN, spinning, and aspirating the NETN solution.

The amount of luciferase activity associated with the GSH beads directly reflected the amount of GST-PPAR-LBD/GW2331 ligand/SRC-Luc complex that was formed. The luciferase activity was quantified as follows: beads were resuspended in 20 μL Promega cell culture lysis buffer and transferred to a white 96 well plate. 5 μL Tris-HCl (pH 9.3) and 100 μL of Promega luciferin mix were added and light emission read with a Dynatech luminometer (DYNEX Technologies, Inc). Luciferase activity was expressed as light units.

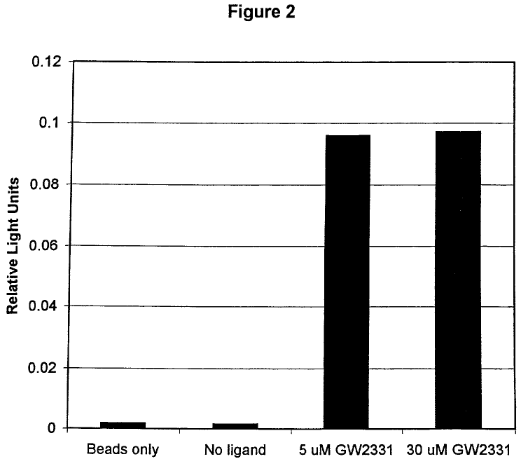

As can be seen in FIG. 2, the assay of the current invention allows for the accurate quantification of GST-PPARα-LBD/GW2331 ligand/SRC-Luc complexes isolated from the in vitro reaction cocktails. As the concentration of GW2331 ligand was increased in each reaction cocktail, the amount of GST-PPARα-LBD/GW2331 ligand/SRC-Luc complex also increased (FIG. 2).

### EXAMPLE 2

### An in vitro PPARβ/SRC-1 Binding Assay

A binding assay was performed in substantially the same manner as described in EXAMPLE 1 above except GST-PPARβ-LBD and the aforementioned PPARβ ligand Compound A were also included in separate in vitro reaction cocktails. The expression plasmid for GST-PPARβ-LBD was prepared as follows: the human PPARβ ligand binding domain including the hinge region (amino acids 136-441, Genbank Accession No. L07592) was PCR amplified from HepG2 first strand cDNA using synthetic primers (SEQ ID:5 and SEQ ID:6) and native Taq polymerase. The primers were tagged with BamHI (5′) and NotI (3′) restriction sites to facilitate cloning. PCR fragments were cleaved with NotI and BamHI and cloned into pGEX-4T-3 (Amersham Pharmacia Biotech) cut with the same enzymes, screened by diagnostic restriction digests, and confirmed by DNA sequence analysis.

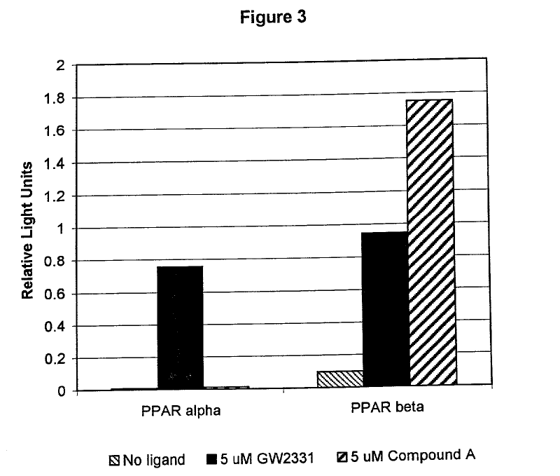

Consistent with EXAMPLE 1 above, GST-PPARα-LBD bound GW2331 efficiently and was simply and accurately quantified (FIG. 3). GST-PPARα-LBD did not, however, bind Compound A. GST-PPARβ-LBD bound GW2331 with roughly equal efficiency as GST-PPARβ-LBD, but bound Compound A with even greater efficiency (FIG. 3).

This example shows that the assays provided by the invention are not limited to a single nuclear receptor but in fact may be used to assay the binding of many nuclear receptors to their coregulators. Moreover, this example shows that the efficiency of binding of any given ligand to multiple nuclear receptors or the efficiency of binding of any given nuclear receptor to multiple ligands can be accurately quantified. The assays provided by the invention therefore provide powerful tools for identifying, e.g., new pharmaceutical agents.

What is claimed is:

1. A method for the determination of the functional effect of a test agent on a nuclear receptor protein or an active fragment thereof comprising the steps of: (A) combining in a first in vitro reaction cocktail said nuclear receptor protein or said active fragment thereof fused to a purification facilitating compound; a nuclear receptor coregulator protein or an active fragment thereof fused to an enzyme or a fragment thereof whose activity is simply quantified; a ligand for said nuclear receptor protein; a purification facilitating partner affixed to a solid support; and said test agent; (B) incubating the components of step (A) to allow said components to form a complex; (C) removing said solid support from the remainder of said first in vitro reaction cocktail; (D) determining an amount of said complex that was formed by assaying said removed solid support for a first activity of said enzyme or fragment thereof; and (E) assessing whether said test agent functioned as an effector of complex formation by comparing said first activity to a second activity from said enzyme or fragment thereof, recovered from a second in vitro reaction cocktail comprising all of the components of step (A) except said test agent, where said second in vitro reaction cocktail was subjected to steps (B) to (D).

2. The method as defined in claim 1 wherein said test agent is a protein, peptide, nucleic acid, hormone, cytokine, lipid, carbohydrate, vitamin, mineral, large organic molecule, small organic molecule, non-organic agent or any combination thereof.

3. The method as defined in claim 1 wherein said nuclear receptor is a steroid receptor.

4. The method as defined in claim 1 wherein said nuclear receptor is a non-steroid receptor.

5. The method as defined in claim 1 wherein said nuclear receptor is a peroxisome proliferator-activated receptor, thyroid receptor, estrogen receptor, glucocorticoid receptor, progesterone receptor, androgen receptor, mineralcorticoid receptor, retinoic acid receptor, retinoid X receptor, vitamin D receptor, orphan receptor, any fragment thereof or any combination thereof.

6. The method as defined in claim 1 wherein said active fragment of said nuclear receptor comprises the ligand binding domain.

7. The method as defined in claim 5 wherein said active fragment of said nuclear receptor comprises the ligand binding domain.

8. The method as defined in claim 1 wherein said active fragment of said nuclear receptor coregulator comprises one or more LXXLL motifs.

9. The method as defined in claim 1 wherein said purification facilitating compound is glutathione-S-transferase, maltose K, influenza hemaglutinin, avidin, biotin, FLAG, myc tag, histidine multimers, or any combination thereof.

10. The method as defined in claim 1 wherein said purification facilitating partner is glutathione, maltose, anti-influenza hemaglutinin antibodies, avidin, biotin, anti-FLAG antibodies, anti-myc antibodies, ionic nickel, or any combination thereof.

11. The method as defined in claim 1 wherein said nuclear receptor coregulator is a nuclear receptor coactivator.

12. The method as defined in claim 11 wherein said nuclear receptor coactivator is a steroid receptor coactivator-1, steroid receptor coactivator-2, steroid receptor coactivator-3, transcription intermediary factor 2, glucocorticoid receptor interacting protein 1, retinoic acid receptor interacting protein 3, coactivator-associated arginine methyltransferase 1, peroxisome proliferator-activated receptor gamma coactivator-1, peroxisome proliferator-activated receptor gamma coactivator-2, p300/CREB binding protein, p300, CREB-binding protein-interacting protein, nuclear-receptor co-activator protein, p300/CBP-associated factor, alteration/deficiency in activation 3 protein, small nuclear RING finger protein, thyroid hormone receptor-associated protein 220, NR-binding SET-domain-containing protein, any fragment thereof, or any combination thereof.

13. The method as defined in claim 1 wherein said nuclear receptor coregulator is a nuclear receptor corepressor.

14. The method as defined in claim 13 wherein said nuclear receptor corepressor is nuclear receptor corepressor (N-Cor), small ubiquitous nuclear corepressor, silencing mediator for retinoic acid and thyroid hormone receptors, transcription intermediary factor 2, thyroid hormone receptor uncoupling protein, calreticulin, repressor of estrogen receptor activity, NR-binding SET-domain-containing protein, any fragment thereof, or any combination thereof.

15. The method as defined in claim 1 wherein said solid support is a glass bead, cellulose bead, polystyrene bead, sephadex bead, sepharose bead, polyacrylamide bead, agarose bead, magnetic bead, multi-well plate, glass reaction vessel, or plastic reaction vessel.

16. The method as defined in claim 1 wherein said enzyme is luciferase, β-galactosidase, alkaline phosphatase, peroxidase, chloramphenicol acetyl transferase or green fluorescent protein.

17. The method as defined in claim 1 wherein said second in vitro reaction cocktail comprises a control agent known to have an effect on said nuclear receptor.

18. The method as defined in claim 5 wherein said second in vitro reaction cocktail comprises a control agent known to have an effect on said nuclear receptor.

19. The method as defined in claim 1 wherein said first in vitro reaction cocktail comprises multiple nuclear receptors or active fragments thereof essentially simultaneously and said method comprises an additional step (f) comprising deconvoluting the active nuclear receptor after assessing whether said test agent functioned as an effector of the nuclear receptor protein.

20. The method as defined in claim 1 wherein said test agent functioned as an agonist of complex formation, an antagonist of complex formation, or a ligand for said nuclear receptor protein.

21. A high-throughput assay comprising the method as defined in claim 1 and utilizing multiple in vitro reaction cocktails for the determination of the functional effect of multiple test agents on a nuclear receptor protein or a fragment thereof.

22. A method for the identification of a nuclear receptor ligand, comprising the steps of: (A) combining in a first in vitro reaction cocktail a nuclear receptor protein or an active fragment thereof fused to a purification facilitating compound; a nuclear receptor coregulator protein or an active fragment thereof fused to an enzyme or a fragment thereof whose activity is simply quantified; a purification facilitating partner affixed to a solid support; and a test agent; (B) incubating the components of step (A) to allow the components to form a complex; (C) removing said solid support from the remainder of said first in vitro reaction cocktail; (D) determining an amount of complex that was formed by assaying said removed solid support for a first activity of the enzyme or fragment thereof; and (E) assessing whether said test agent functioned as a ligand for the nuclear receptor or active fragment thereof by comparing said first activity to a second activity from said enzyme or fragment thereof recovered from a second in vitro reaction cocktail comprising all of the components of step (A) except said test agent, where said second in vitro reaction cocktail was subjected to steps (B) to (D).

23. The method as defined in claim 22 wherein said test agent is a protein, peptide, nucleic acid, hormone, cytokine, lipid, carbohydrate, vitamin, mineral, large organic molecule, small organic molecule, non-organic agent or any combination thereof.

24. The method as defined in claim 22 wherein said nuclear receptor is a steroid receptor.

25. The method as defined in claim 22 wherein said nuclear receptor is a non-steroid receptor.

26. The method as defined in claim 22 wherein said nuclear receptor is peroxisome proliferator-activated receptor, thyroid receptor, estrogen receptor, glucocorticoid receptor, progesterone receptor, androgen receptor, mineralcorticoid receptor, retinoic acid receptor, retinoid X receptor, vitamin D receptor, orphan receptor, any fragment thereof, or any combination thereof.

27. The method as defined in claim 22 wherein said active fragment of said nuclear receptor comprises the ligand binding domain.

28. The method as defined in claim 26 wherein said active fragment of said nuclear receptor comprises the ligand binding domain.

29. The method as defined in claim 22 wherein said active fragment of said nuclear receptor coregulator comprises one or more LXXLL motifs.

30. The method as defined in claim 22 wherein said purification facilitating compound is glutathione-S-transferase, maltose K, influenza hemaglutinin, avidin, biotin, FLAG, myc tag or histidine multimers.

31. The method as defined in claim 22 wherein said purification facilitating partner is glutathione, maltose, anti-influenza hemaglutinin antibodies, avidin, biotin, anti-FLAG antibodies, anti-myc antibodies, ionic nickel, or any combination thereof.

32. The method as defined in claim 22 wherein said nuclear receptor coregulator is a nuclear receptor coactivator.

33. The method as defined in claim 32 wherein said nuclear receptor coactivator is steroid receptor coactivator-1, steroid receptor coactivator-2, steroid receptor coactivator-3, transcription intermediary factor 2, glucocorticoid receptor interacting protein 1, retinoic acid receptor interacting protein 3, coactivator-associated arginine methyltransferase 1, peroxisome proliferator-activated receptor gamma coactivator-1, peroxisome proliferator-activated receptor gamma coactivator-2, p300/CREB binding protein, p300, CREB-binding protein-interacting protein, nuclear-receptor co-activator protein, p300/CBP-associated factor, alteration/deficiency in activation 3 protein, small nuclear RING finger protein, thyroid hormone receptor-associated protein 220, NR-binding SET-domain-containing protein, any fragment thereof, or any combination thereof.

34. The method as defined in claim 22 wherein said nuclear receptor coregulator is a nuclear receptor corepressor.

35. The method as defined in claim 34 wherein said nuclear receptor corepressor is nuclear receptor corepressor (N-Cor), small ubiquitous nuclear corepressor, silencing mediator for retinoic acid and thyroid hormone receptors, transcription intermediary factor 2, thyroid hormone receptor uncoupling protein, calreticulin, repressor of estrogen receptor activity, NR-binding SET-domain-containing protein, any fragment thereof or any combination thereof.

36. The method of claim 22 wherein said solid support is a glass bead, cellulose bead, polystyrene bead, sephadex bead, sepharose bead, polyacrylamide bead, agarose bead, magnetic bead, multi-well plate, glass reaction vessel or plastic reaction vessels.

37. The method of claim 22 wherein said enzyme is luciferase, β-galactosidase, alkaline phosphatase, peroxidase, chloramphenicol acetyl transferase, and green fluorescent protein.

38. The method as defined in claim 22 wherein said second in vitro reaction cocktail comprises a control agent known to be a ligand for said nuclear receptor or active fragment thereof.

39. A high-throughput assay comprising the method as defined in claim 22 and utilizing multiple in vitro reaction cocktails for the identification of a ligand for a nuclear receptor protein or a fragment thereof.

40. A method for the determination of a functional effect of a test agent on a peroxisome proliferator-activated receptor protein or a fragment thereof, comprising the steps of: (A) combining in a first in vitro reaction cocktail said peroxisome proliferator-activated receptor protein or said fragment thereof fused to the glutathione-S-transferase protein or a fragment thereof; a steroid receptor coactivator-1 protein or a fragment thereof fused to the luciferase protein or a fragment thereof; a GW2331 ligand; glutathione-sepharose beads; and said test agent; (B) incubating the components of step (A) to allow said components to form a complex; (C) removing said glutathione-sepharose beads from the remainder of said first in vitro reaction cocktail; (D) determining an amount of said complex that was formed by assaying said removed glutathione-sepharose beads for a first luciferase activity; and (E) assessing whether said test agent functioned as an effector of complex formation by comparing said first luciferase activity to a second luciferase activity recovered from a second in vitro reaction cocktail comprising all of the components of step (A) except said test agent, where said second in vitro reaction cocktail was subjected to steps (B) to (D).

41. A method for the identification of a ligand for a peroxisome proliferator-activated receptor protein or a fragment thereof, comprising the steps of: (A) combining in a first in vitro reaction cocktail said peroxisome proliferator-activated receptor protein or said fragment thereof fused to the glutathione-S-transferase protein or a fragment thereof; a steroid receptor coactivator-1 protein or a fragment thereof fused to the luciferase protein or a fragment thereof; glutathione-sepharose beads; and a test agent; (B) incubating the components of step (A) to allow said components to form a complex; (C) removing said glutathione-sepharose beads from the remainder of said first in vitro reaction cocktail; (D) determining an amount of said complex that was formed by assaying said removed glutathione-sepharose beads for a first luciferase activity; and (E) assessing whether said test agent functioned as a ligand for peroxisome proliferator-activated receptor protein by comparing said first luciferase activity to a second luciferase activity recovered from a second in vitro reaction cocktail comprising all of the components of step (A) except said test agent, where said second in vitro cocktail was subjected to steps (B) to (D).

42. A pharmaceutical composition comprising an effector as defined in claim 1 and a pharmaceutically acceptable carrier, vehicle, or diluent.
